(19) United States
(12) Patent Application Publication
(10) Pub. No.: US 2006/0173493 A1
(43) Pub. Date: Aug. 3, 2006

(54) Multi-phasic signal for stimulation by an implantable device

(75) Inventors: Randolph K. Armstrong, Houston, TX (US); Scott A. Armstrong, Danbury, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: Cyberonics, Inc.

(21) Appl. No.: 11/046,430

(22) Filed: Jan. 28, 2005

(51) Int. Cl. A61N 1/00

(52) U.S. Cl. 607/2; 607/45

(57) ABSTRACT

A method, system, and an apparatus for providing a multi-phasic stimulation signal for an implantable device are provided. An electrical pulse with a first characteristic that includes a first pulse width, a first pulse amplitude, a first pulse polarity, or a first pulse shape, is applied during a first time period to a portion of a vagus nerve using an implantable device. A controlled modification of the first characteristic of the electrical pulse is performed. The controlled modification is performed to provide a second characteristic for the electrical pulse during a second time period. The electrical pulse with the second characteristic is applied to the target portion of the vagus nerve.

### BACKGROUND OF THE INVENTION

1. Field of the Invention

This invention relates generally to implantable medical devices, and, more particularly, to methods, apparatus, and systems for providing a multi-phasic pulse signal for stimulation of biological tissue by an implantable medical device.

2. Description of the Related Art

There have been many improvements over the last several decades in medical treatments for disorders of the nervous system, such as epilepsy and other motor disorders, and abnormal neural discharge disorders. One of the more recently available treatments involves the application of an electrical signal to reduce various symptoms or effects caused by such neural disorders. For example, electrical signals have been successfully applied at strategic locations in the human body to provide various benefits, including reducing occurrences of seizures and/or improving or ameliorating other conditions. A particular example of such a treatment regimen involves applying an electrical signal to the vagus nerve of the human body to reduce or eliminate epileptic seizures, as described in U.S. Pat. No. 4,702,254 to Dr. Jacob Zabara, which is hereby incorporated in its entirety herein by reference in this specification. Electrical stimulation of the vagus nerve (hereinafter referred to as vagus nerve stimulation therapy or VNS) may be provided by implanting an electrical device underneath the skin of a patient and performing a detection and electrical stimulation process. Alternatively, the system may operate without a detection system once the patient has been diagnosed with epilepsy, and may periodically apply a series of electrical pulses to the vagus (or other cranial) nerve intermittently throughout the day, or over another predetermined time interval.

Many types of implantable medical devices, such as pacemakers and drug infusion pumps, typically include custom integrated circuits that are complex, expensive, and specific to the intended use. These systems also typically employ proprietary communication techniques to transfer information between the implant and an external programmer. The custom circuitry is developed because of the need to keep power consumption at a minimum, to conform to the allowable size for implantable devices, and to support the complexity of the detection and communication techniques, while still supplying the particular intended therapy.

State of the art implantable neurostimulator devices generally provide a burst of substantially uniform electrical pulses. Some patients may experience a therapeutic benefit from a uniform-pulse treatment regiment, while other patients may not. Generally, current pulses provided by state of the art implantable devices include a constant current pulse having programmable parameters such as current magnitude, pulse width, frequency, on-time (i.e., how long a stimulation period continues), and off-time (i.e., the length of time between stimulation periods). The pulses are delivered for the programmed on-time period, and then turned off for the programmed off-time period. The ratio of on-time to off-time is sometimes referred to as the duty cycle of the neurostimulator. State-of-the-art implantable devices generally deliver constant current pulses according to the programmed duty cycle or in response to manual initiation of the therapy by the patient or a caregiver.

For many patients not initially responding to neurostimulation therapy such as VNS therapy, altering the therapy to provide another type of pulse may provide therapeutic benefit. State-of-the-art implantable neurostimulators generally only provide a single type of pulse signal that has a single phase.

A nerve bundle to which neurostimulation therapy is applied may comprise up to 100,000 or more individual nerve fibers of different types, including larger diameter A and B fibers which comprise a myelin sheath and C fibers which have a much smaller diameter and are unmyelinated. Different types of nerve fibers respond differently to different types of stimulation signals. These different responses among nerve fiber types reflect, among other things, their different sizes, conduction velocities, stimulation thresholds, and myelination status (i.e., myelinated or unmyelinated). Therefore, depending on which type(s) of nerve fibers are the target of the stimulation therapy, different responses by the patient's body occur. In general, the larger, myelinated A and B fibers have a lower stimulation threshold than the unmyelinated, smaller C fibers. Thus, while it is possible to selectively stimulate A and/or B fibers to generate an action potential without generating an action potential in the C fibers, it is not possible currently to stimulate C fibers without also generating an action potential in the A and B fibers. Accordingly, the constant current pulses provided by state-of-the-art implantable neurostimulators are generally incapable of performing selective activation or selective inhibition of any desired type of fiber within a nerve bundle. Although considerable efforts have been made to target and stimulate specific regions of a patient's body, state-of-the-art implantable neurostimulators generally have not been sophisticated enough to provide much more than a uniform pulse signal to provide neural stimulation.

Current neurostimulators also provide the potential for a number of post-implantation problems. For example, the electrodes associated with a neurostimulator typically require a particular orientation on a nerve fiber. For example, VNS therapy generally requires that the negative electrode (i.e., the cathode) be placed proximal to the brain along the vagus nerve bundle relative to the positive electrode to achieve therapeutic efficacy. If the electrodes are implanted in the reverse order, correction of this error may require further surgery that imposes additional physical hardship upon a patient, economic costs, loss of time, etc. State-of-the-art neurostimulators also typically lack an efficient means for providing flexibility in stimulation techniques to adapt stimulation therapy according to the patient's response to the therapy. Certain nerve fibers, for example, may physically change in response to the initiation of neurostimulation therapy such that uniform-type signals may cease to be therapeutically effective over time. Greater flexibility in the types of signals deliverable by neurostimulators would be a desirable feature that is not available in state-of-the-art devices.

The present invention is directed to overcoming, or at least reducing, the effects of one or more of the problems set forth above.

### SUMMARY OF THE INVENTION

In one aspect, the present invention comprises a method of treating a patient with a multi-phasic stimulation signal from an implantable medical device (IMD). An electrical pulse with a first characteristic that includes a first pulse width, a first pulse amplitude, a first pulse polarity, and/or a first pulse shape, is applied during a first time period to a portion of a vagus nerve using an implantable device. A controlled modification of the first characteristic of the electrical pulse is performed. The controlled modification is performed to provide a second characteristic for the electrical pulse during a second time period. The electrical pulse with the second characteristic is applied to the target portion of the vagus nerve.

In another aspect, the method comprises providing an electrical pulse with a first characteristic. The first characteristic may be a pulse width, a pulse amplitude, a pulse polarity, and/or a pulse shape. The electrical pulse is applied to a target portion of a vagus nerve during a first phase relating to a first time period. A controlled modification of the first characteristic of the electrical pulse is performed during a second phase relating to a second time period to provide a second characteristic for the electrical pulse during a second time period. The electrical pulse with the second characteristic is applied during the second phase to the target portion of the vagus nerve.

In a further aspect, the method comprises providing an electrical pulse with a first pulse width, a first pulse amplitude, a first pulse polarity, and/or a first pulse shape. The electrical pulse is provided during a first phase associated with a first time period to a target portion of a vagus nerve using the IMD. The electrical pulse is applied during the first phase to the target portion of the vagus nerve. A controlled modification is performed upon the electrical pulse to have a second pulse width, a second pulse amplitude, a second pulse polarity, and/or second pulse shape during a second phase associated with a second time period. The electric pulse is applied during the second phase to the target portion of the vagus nerve.

In another aspect of the present invention, an implantable medical device is provided for delivering a multi-phasic stimulation signal to a patient. The IMD comprises a stimulation unit to provide an electrical pulse during a first time period to a target portion of a vagus nerve. The electrical pulse has a first characteristic during the first time period. The first characteristic includes a first amplitude, a first polarity, a first pulse width, and/or first pulse shape. The IMD also comprises a controller operatively coupled to the stimulation unit. The controller is adapted to direct the stimulation unit to apply the pulse that has the first characteristic, to the target portion of the vagus nerve. The controller is also adapted to perform a controlled modification of the first characteristic to generate a pulse with a second characteristic during a second time period. The second characteristic includes a second amplitude, a second polarity, a second pulse width, and/or a second pulse shape.

In yet another aspect, the present invention comprises a computer readable program storage device encoded with instructions for providing a multi-phasic stimulation signal for an implantable medical device. The instructions in the computer readable program storage device, when executed by a computer, apply an electrical pulse with a first characteristic that includes a pulse width, a pulse amplitude, a pulse polarity, and/or a pulse shape, to a target portion of a vagus nerve using a pulse generator in a first time period. The instructions, when executed by a computer, may also perform a controlled modification of the first characteristic of the electrical pulse to provide a second characteristic for the electrical pulse during a second time period; and apply the electrical pulse with the second characteristic to the target portion of the vagus nerve.

In yet another aspect of the present invention, a method is provided for treating a patient with a tri-phasic stimulation signal from an IMD. The method of the present invention includes applying an electrical pulse to a target portion of a cranial nerve using a pulse generator. Applying the electrical signal includes applying an electrical pulse that includes a first phase corresponding to a first characteristic, a second phase corresponding to a second characteristic, and a third phase corresponding to a third characteristic,. The first characteristic comprises at a first amplitude, a first polarity, a first pulse width, and/or a first pulse shape. The second characteristic includes a second amplitude, a second polarity, a second pulse width, and/or a second pulse shape. The third characteristic includes a third amplitude, a third polarity, a third pulse width, and/or a third pulse shape.

In another aspect of the present invention, an IMD is provided for delivering a multi-phasic stimulation signal to a patient. The IMD includes a stimulation unit to provide an electrical pulse train during to a target portion of a vagus nerve. The electrical pulse train includes a first pulse having a first characteristic during a first time period. The first characteristic comprising a first amplitude, a first polarity, a first pulse width, and/or a first pulse shape. The IMD also includes a controller operatively coupled to the stimulation unit. The controller is adapted to direct the stimulation unit to apply the pulse train. That has the first pulse to the target portion of the vagus nerve The controller is also adapted to perform a controlled modification of the first pulse to generate a second pulse with a second characteristic during a second time period. The second characteristic includes a second amplitude, a second polarity, a second pulse width, and/or a second pulse shape.

In another aspect of the present invention, an IMD is provided for delivering a tri-phasic stimulation signal to a patient. The IMD includes a stimulation unit for providing an electrical pulse that includes a first phase corresponding to a first characteristic, a second phase corresponding to a second characteristic, and a third phase corresponding to a third characteristic, to a target portion of a cranium nerve. The IMD also includes a controller operatively coupled to the stimulation unit. The controller is adapted to direct the stimulation unit to apply the electrical signal to the target portion of the cranium nerve.

In yet another aspect, the present invention comprises a computer readable program storage device encoded with instructions for providing a tri-phasic stimulation signal for an IMD. The instructions in the computer readable program storage device, when executed by a computer, apply an electrical pulse to a target portion of a cranial nerve using a pulse generator. The instruction when executed by a computer may also apply an electrical pulse that includes a first phase corresponding to a first characteristic, a second phase corresponding to a second characteristic, and a third phase corresponding to a third characteristic,. The first characteristic includes a first amplitude, a first polarity, a first pulse width, and/or a first pulse shape. The second characteristic includes a second amplitude, a second polarity, a second pulse width, and/or a second pulse shape. The third characteristic includes a third amplitude, a third polarity, a third pulse width, and/or a third pulse shape.

### BRIEF DESCRIPTION OF THE DRAWINGS

The invention may be understood by reference to the following description taken in conjunction with the accompanying drawings, in which like reference numerals identify like elements, and in which:

FIG. 1A is a stylized diagram of a mono-phasic controlled current pulse signal that may be delivered by an implantable medical device;

FIG. 1B is a stylized diagram of a multi-phasic controlled current pulse signal that may be delivered by an implantable medical device, in accordance with an illustrative embodiment of the present invention;

FIG. 1C is a stylized diagram of a multi-phasic controlled current pulse signal that may be delivered by an implantable medical device, in accordance with an alternative illustrative embodiment of the present invention;

FIG. 1D is a stylized diagram of an implantable medical device implanted into a patient's body for providing stimulation to a vagus nerve, in accordance with one illustrative embodiment of the present invention;

FIG. 2 is a block diagram of an implantable medical device and an external unit that communicates with the implantable medical device, in accordance with one illustrative embodiment of the present invention;

FIG. 3 is a diagram of a uniform-type output current signal that may be provided by the implantable medical device of FIGS. 1 and 2;

FIGS. 4A-4F are diagrams of various multi-phasic output current signals provided by the implantable device of FIGS. 1 and 2, in accordance with various illustrative embodiments of the present invention;

FIG. 5 is a more detailed block diagram of a stimulation controller of FIG. 2, in accordance with one illustrative embodiment of the present invention;

FIG. 6 is a more detailed block diagram of a switching network of FIG. 5, in accordance with one illustrative embodiment of the present invention;

FIG. 7 is a more detailed block diagram of a current source of FIG. 5, in accordance with one illustrative embodiment of the present invention;

FIG. 8 provides a stylized depiction of an implantable medical device and its leads coupled to a target portion of a patient's body, in accordance with one illustrative embodiment of the present invention;

FIG. 9 is a flowchart representation of a method of performing a multi-phasic stimulation using an implantable medical device, in accordance with one illustrative embodiment of the present invention; and

FIG. 10 is a flowchart representation of a method of using a phasic pulse description to implement the multi-phasic stimulation, in accordance with one illustrative embodiment of the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

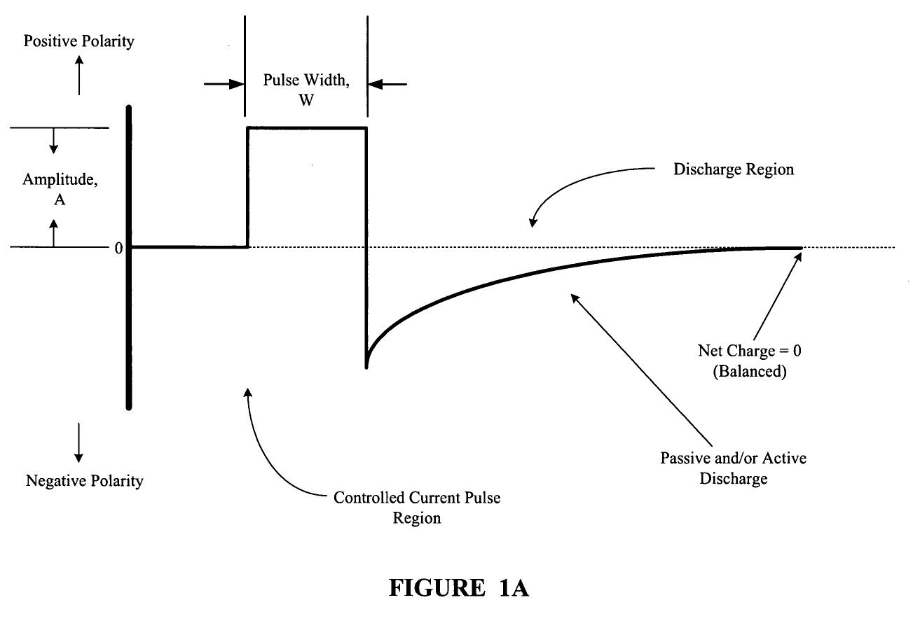

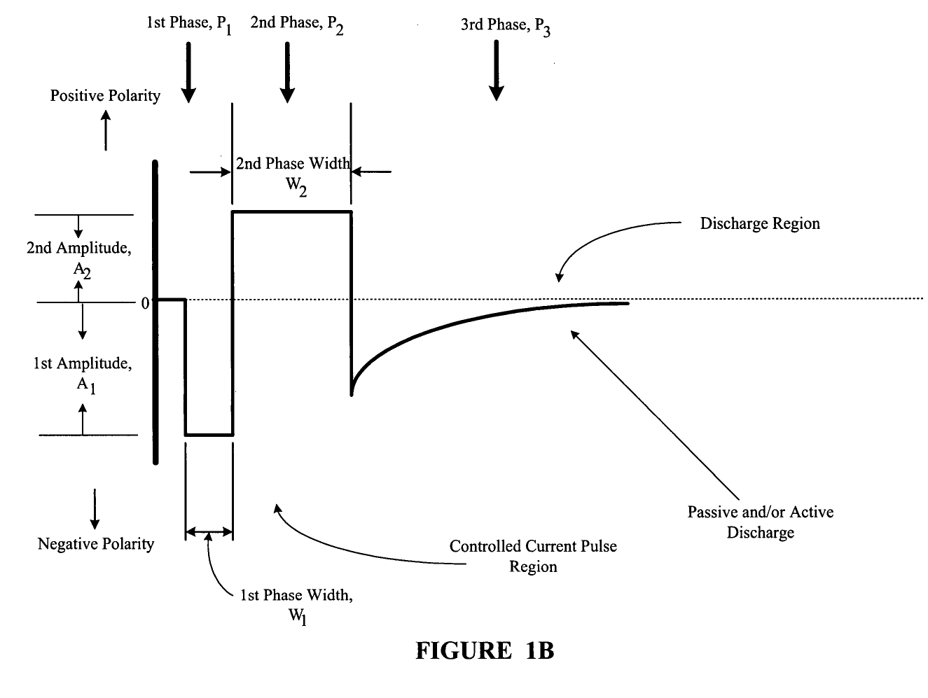

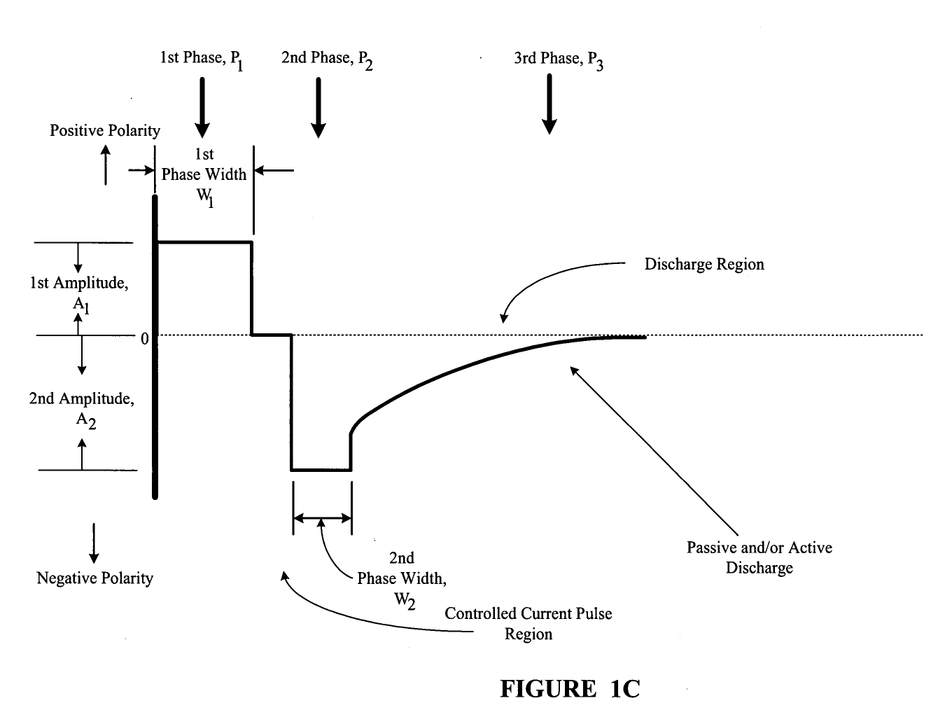

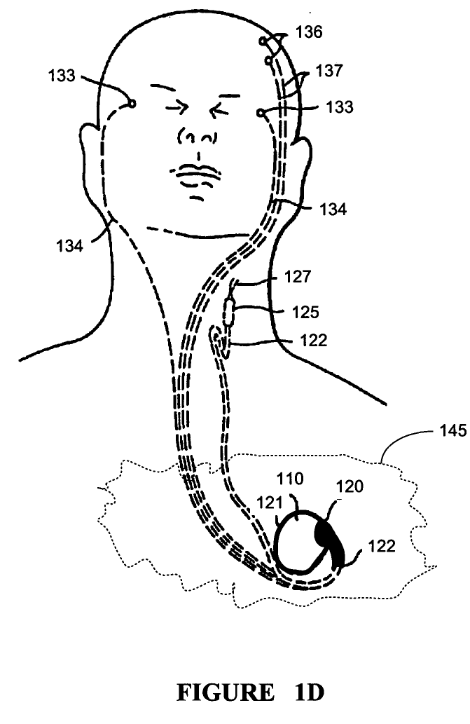

In one embodiment of the present invention, methods, apparatuses, and systems for providing a multi-phasic controlled current pulse to perform a tissue/nerve stimulation of a portion of a human body, e.g., stimulating the vagus nerve in a human body. Turning now to FIG. 1A, a diagram illustrating a mono-phasic controlled current pulse is provided. The mono-phasic controlled current pulse has a controlled current amplitude, A. The pulse signal of FIG. 1A may be used to stimulate a tissue or nerve portion of a human body via a lead and an electrode that is electrically coupled to the tissue or nerve portion. The pulse of FIG. 1A has a pulse width, W. Upon completion of the pulse signal, a discharge stage occurs, as illustrated in FIG. 1A. Discharging of the energy due to the pulse is performed after the controlled current pulse, in order to balance the charge delivered. The discharge of the energy of the pulse signal may be performed in an active or in a passive manner. Passive discharge refers to discharging energy using discharging balancing capacitors through a lead resistance and/or tissue resistance. Active discharge refers to discharging energy using the passive means described above as well as using internally-added resistance in an implantable medical device.

One benefit of using controlled current signals to stimulate tissue/nerve portions of a human body instead of voltage stimulation is that the electrical field experienced by excitable tissue is independent of the impedance of the tissue/nerve portion and of the electrode.

Turning now to FIG. 1B, a diagram depicting a multi-phasic controlled current pulse signal is illustrated. In one embodiment, multi-phasic signals generally refer to multiple periods of time associated with a pulse signal in which a controlled current may be delivered, wherein the controlled current has a delivery characteristic that is different from a preceding or subsequent period of time. In one embodiment, a multi-phase pulse signal may refer to multiple phases associated with a particular pulse. In this embodiment, a first phase of a pulse is provided in a first time period, wherein the first phase of the pulse signal may be characterized by a first characteristic. The first characteristic may include a first pulse width, a first pulse amplitude, a first pulse polarity, and/or a first pulse shape. Similarly a second phase of the pulse may be provided in a second time period, wherein the second phase of the pulse may be characterized by a second characteristic. The second characteristic may include a second pulse width, a second pulse amplitude, a second pulse polarity, and/or a second pulse shape. Additionally, a third phase of the pulse may be provided in a third time period, wherein the third phase of the pulse may be characterized by a third characteristic. The third characteristic may include a third pulse width, a third pulse amplitude, a third pulse polarity, and/or a third pulse shape. One or more of the characteristic may be modified in any given phase.

In an alternative embodiment, a multi-phase signal may refer to a plurality of phases associated with a plurality of corresponding pulses in a pulse train. Each phase in the alternative embodiment may be characterized by different characteristics, such as different pulse widths, different pulse amplitudes, different pulse polarities, and/or a different pulse shapes. The modifications to the pulse characteristics relating to the embodiments described above may be performed in a controlled manner, e.g., controlled current pulses. The embodiments described in the present invention are capable of performing a frequency sweep across a wide range of frequencies. Therefore, different phases may be characterized by varied frequencies. In an alternative embodiment, a random frequency change (i.e., non-controlled frequency change) in the phases may be implemented by embodiments of the present invention.

For clarity and ease of description, FIG. 1B illustrates a signal depicting three phases; however, a multi-phasic controlled current signal may comprise any number of phases that may each contain pulse-characteristics of different shapes, amplitude, polarity, phase-width, or the like. The first phase, P₁, of the signal shown in FIG. 1B may comprise a first amplitude A₁, a first phase-width, W₁, and a negative polarity. The second phase, P₂, of the signal shown in FIG. 1B may comprise a second amplitude A₂, a second phase-width, W₂, and a positive polarity. A third phase, P₃, may be a discharge phase, as illustrated in FIG. 1B. In one embodiment, the discharge may be implemented in a passive manner. In an alternative embodiment, the discharge phase may be implemented in an active manner, wherein the discharging of the energy relating to the phases pervious to the discharge phase may be performed in a controlled, active manner. In yet an alternative embodiment, the discharging of the energy relating to the phases pervious to the discharge phase may be performed in a combination of an active and a passive manner. In one embodiment, the term “multi-phasic” may refer to a bi-phasic signal, which may comprise a first phase and a second phase. In an alternative embodiment, the term “multi-phasic” may refer to a tri-phasic signal, which may comprise a first phase, a second phase, and a third phase. In yet another embodiment, the term “multi-phasic” may refer to a signal with any number of phases.

As illustrated in FIG. 1B, the first, second, and third phases (P₁, P₂, P₃) correspond to regions that have different pulse characteristics. For example, the first and second amplitudes (A₁, A₂) are different, the first and second phase-widths (W₁, W₂) are different, and the polarity of the respective portions of the pulses corresponding to the first and second phases may also be different. The third phase may be a charge balance phase, wherein charge/energy resulting from the first and second phases may be discharged. Additionally, variations within each of the pulses may be effectuated when delivering the multi-phasic controlled current pulse signal shown in FIG. 1B. Furthermore, FIG. 1B illustrates a discharge region. The discharging of the energy of the signal may be performed in an active manner, a passive manner, and/or in a combination thereof. The multi-phasic pulse signal may refer to signal variations within a particular pulse. For example, the amplitude of a particular pulse may change during the duration of the pulse, wherein the time period where a first amplitude of the pulse may occur in a first phase and a second amplitude of the same pulse may occur in a second phase. This provides for a plurality of independent phases within a particular pulse. More detailed descriptions of the multi-phasic pulse signals are provided below.

Referring to FIG. 1C, an exemplary alternative embodiment of a multiphase signal response in accordance with embodiments of the present invention is illustrated. FIG. 1C illustrates an exemplary tri-phase signal that may be provided by an implantable medical device. The first phase, P₁, of the signal illustrated in FIG. 1C comprises a first amplitude, A₁. In one embodiment, the first phase comprises a pulse width W₁. The first phase P₁ may be provided to perform a therapeutic stimulation of a portion of a human body. Subsequent to the first phase, a second phase P₂ may be provided, wherein the second phase P₁ comprises a second amplitude A₂ and a second phase width W₂. In one embodiment, the second phase P₂ may be provided to perform a de-polarizing function in order to isolate a particular portion of a human body from the effects of the therapeutic pulse provided in the first phase P₁. The third phase P₃ may be a discharge phase, which may be an active and/or a passive discharge to discharge the energy and/or to balance the energy generated from the activation of the first and second phases. In one embodiment, the pulses provided in the various phases described in FIGS. 1A-1C may be implemented in a variety of control fashions, such as analog controlled current signals, digitally current control signals, and/or control current signals resulting from firmware control. Additionally, the signal illustrated in FIGS. 1A-1C may be controlled in an arbitrary fashion wherein a variety of pulses and shapes of the control current signal may be implemented.

Embodiments of the present invention provide methods, apparatus and systems for delivering a multi-phasic controlled current signal for stimulating a target portion of a patient's body using an implantable medical device (IMD). Embodiments of the present invention provide for an IMD capable of providing a plurality of phases for the signal pulses used for stimulation. For example, a stimulation signal may comprise a pulse train (i.e., a series of pulses) wherein a certain number of the pulses have a greater amplitude than others in the series, while another number of pulses in the series may have an opposite polarity from the other pulses in the series. The multi-phasic system provided by embodiments of the present invention provide an IMD to provide a first multiple independent phase in a pulse. Embodiments of the present invention also provide an IMD to provide a second multiple independent pulse in a burst or a pulse train. This pulse train may comprise various pulses of multiple amplitudes, multiple durations of various pulses, different phase widths, and/or multiple polarities of various pulses. In one embodiment, polarities of pulses may be varied for each subsequent phase. Additionally, embodiments of the present invention also allow the shape of one or more pulses of the stimulation signal to be controlled to achieve various advantages, such as performing selective activation of stimulation of a target portion of a patient's body. Embodiments of the present invention may also provide for an IMD to provide a stimulation signal that comprises a pulse that has independent phases as well as a pulse train where variations between the pulses exist. Embodiments of the present invention may also provide the ability to stimulate a plurality of electrodes, wherein each electrode may have different sets of phases for the controlled current signal. In one embodiment, various electrodes may be activated for various phases.

As previously noted, different types of nerve fibers (e.g., A, B, and C fibers being different fibers being targeted for stimulation) respond differently to stimulation from electrical signals. More specifically, the different types of nerve fibers have different conduction velocities, and therefore differ in their responsiveness to stimulation. Certain pulses of an electrical stimulation signal, for example, may be below the stimulation threshold for a particular fiber and therefore may generate no action potential in the fiber. Thus, smaller or narrower pulses may be used to avoid stimulation of certain nerve fibers (such as C fibers) and target other nerve fibers (such as A and/or B fibers). Additionally, techniques such as pre-polarization may be employed wherein particular nerve regions may be polarized before a more robust stimulation is delivered, which may better accommodate particular electrode materials. Furthermore, opposing polarity phases separated by a zero current phase may be used to excite particular axons or postpone nerve fatigue during long-term stimulation.

Embodiments of the present invention allow for creation of more effective stimulation signals, which may be tailored for particular types of nerve fibers or for particular patients. The dynamic current paths (i.e., varying the current path by activating various electrodes), direction (i.e., varying the polarity of the various electrodes) amplitude, frequency, pulse duration, and pulse shapes for stimulation signals provided by embodiments of the present invention may be used to increase efficacy and/or reduce complications via selective activation and/or selective inhibition of particular types of nerve fibers. For example, selective inhibition may be used to provide for afferent selectivity (i.e., stimulation only in the afferent direction while avoiding efferent stimulation), vocal stimulation prevention, Brady/syncope prevention, (i.e., preventing efferent stimulation that may impact cardiac function) and generally targeting particular nerve fibers. Embodiments of the present invention may also reduce the total energy that is required for proper stimulation efficacy. The dynamic polarity techniques for stimulation signals provided by embodiments of the present invention may be used to increase efficacy and/or perform post-implant reversal to reduce complications. Post-implant reversal may relate to reversing the polarity of electrodes in case the leads connecting to various electrodes were installed incorrectly.

Additionally, the multi-phasic signal provided for IMDs in embodiments of the present invention may be used for performing hyper-polarization prior to de-polarization of a particular nerve area, which may be used to allow selective nerve stimulation. The pulse shape provided by embodiments of the present invention may be chosen to hyper-polarize, de-polarize, and/or re-polarize, etc. in order to increase neural conduction or neural inhibition, such as refractory inhibition; as well as to achieve other purposes, such as reduction of stimulation or a reduction of energy required for stimulation or reducing side effects.

FIG. 1D illustrates a generator 110 having a case 121 with an electrically conducting connector 120 implanted in the patient's chest in a pocket or cavity formed by the implanting surgeon just below the skin (as indicated by a dotted line 145), much as a pacemaker pulse generator would be implanted, for example. A stimulating nerve electrode assembly 125, preferably comprising an electrode pair, is conductively connected to the distal end of an insulated electrically conductive lead assembly 122, which preferably comprises a pair of lead wires and is attached at its proximal end to the connector 120 on the case 121. The electrode assembly is surgically coupled to a vagus nerve 127 in the patient's neck. The electrode assembly 125 preferably comprises a bipolar stimulating electrode pair (not shown), such as the electrode pair described in U.S. Pat. No. 4,573,481 issued Mar. 4, 1986 to Bullara. Persons of skill in the art will appreciate that many electrode designs could be used in the present invention. The two electrodes are preferably wrapped about the vagus nerve, and the electrode assembly 125 secured to the nerve 127 by a spiral anchoring tether such as that disclosed in U.S. Pat. No. 4,979,511 issued Dec. 25, 1990 to Reese S. Terry, Jr. and assigned to the same assignee as the instant application. Lead assembly 122 is secured, while retaining the ability to flex with movement of the chest and neck, by a suture connection to nearby tissue.

In one embodiment, the open helical design of the electrode assembly 125 (described in detail in the above-cited Bullara patent), which is self-sizing and flexible, minimizes mechanical trauma to the nerve and allows body fluid interchange with the nerve. The electrode assembly 125 conforms to the shape of the nerve, providing a low stimulation threshold by allowing a large stimulation contact area. Structurally, the electrode assembly 125 comprises two electrode ribbons (not shown), of a conductive material such as platinum, iridium, platinum-iridium alloys, and/or oxides of the foregoing. The electrode ribbons are individually bonded to an inside surface of an elastomeric body portion of two spiral electrodes, which may comprise two spiral loops of a three-loop helical assembly. The lead assembly 122 may comprise two distinct lead wires or a coaxial cable whose two conductive elements are respectively coupled to one of the conductive electrode ribbons. One suitable method of coupling the lead wires or cable to the electrodes comprises a spacer assembly such as that depicted in U.S. Pat. No. 5,531,778 issued Jul. 2, 1996, to Steven Maschino, et al. and assigned to the same Assignee as the instant application, although other known coupling techniques may be used. The elastomeric body portion of each loop is preferably composed of silicone rubber, and the third loop acts as the anchoring tether for the electrode assembly 125.

In certain embodiments of the invention, eye movement sensing electrodes 133 (FIG. 1D) may be implanted at or near an outer periphery of each eye socket in a suitable location to sense muscle movement or actual eye movement. The electrodes 133 may be electrically connected to leads 134 implanted via a catheter or other suitable means (not shown) and extending along the jaw-line through the neck and chest tissue to the stimulus generator 110. The sensing electrodes 133 are utilized for detecting rapid eye movement (REM) in a pattern indicative of a disorder to be treated, as described in greater detail below. Alternatively or additionally, EEG sensing electrodes 136 may be implanted in spaced apart relation through the skull, and connected to leads 137 implanted and extending along the scalp and temple and then along the same path and in the same manner as described above for the eye movement electrode leads. These or other types of sensing electrodes may be used in some embodiments of the invention to trigger administration of the electrical stimulation therapy to the vagus nerve 127 via electrode assembly 125. Use of such sensed body signals to trigger or initiate stimulation therapy is hereinafter referred to as a feedback loop mode of administration. Other embodiments of the present invention utilize a continuous, periodic or intermittent stimulus signal applied to the vagus nerve (each of which constitutes a form of continual application of the signal) according to a programmed on/off duty cycle without the use of sensors to trigger therapy delivery. This type of delivery may be referred to as a prophylactic therapy mode. Both prophylactic and feedback loop administration may be combined or delivered by a single IMD according to the present invention. Either or both modes may be appropriate to treat the particular disorder diagnosed in the case of a specific patient under observation.

The pulse generator 110 may be programmed with an external computer (not shown) using programming software of the type copyrighted by the assignee of the instant application with the Register of Copyrights, Library of Congress, or other suitable software based on the description herein, and a programming wand (not shown) to facilitate radio frequency (RF) communication between the PC and the pulse generator. The wand and software permit noninvasive communication with the generator 110 after the latter is implanted. The wand is preferably powered. by internal batteries, and provided with a “power on” light to indicate sufficient power for communication. Another indicator light may be provided to show that data transmission is occurring between the wand and the generator.

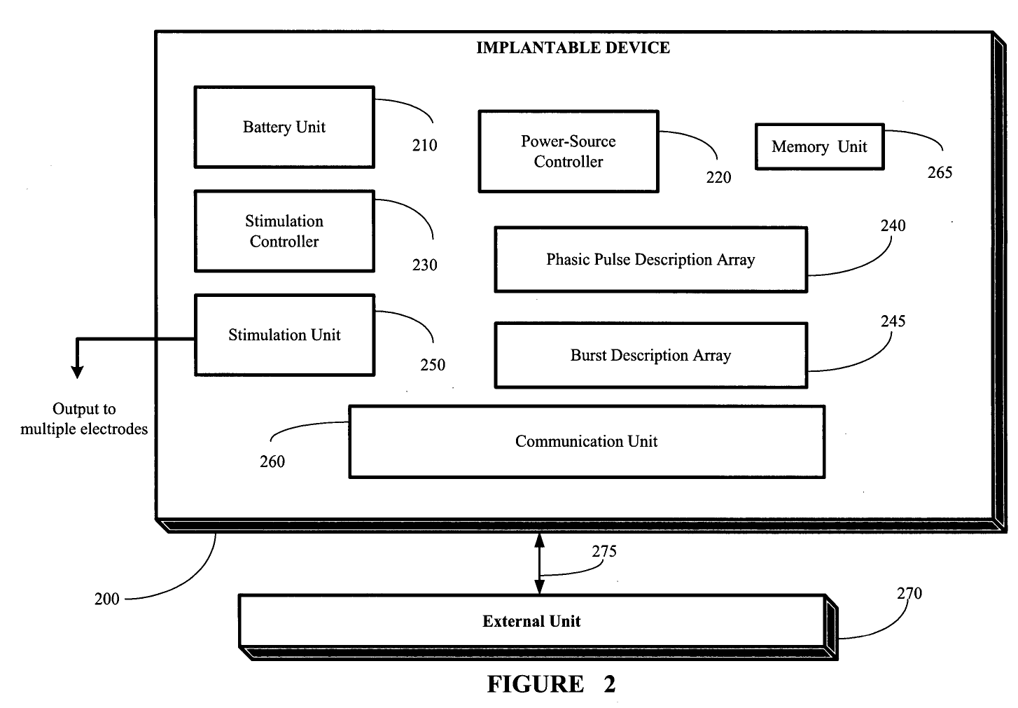

Turning now to FIG. 2, one embodiment of an IMD 200 capable of performing various stimulations, in accordance with embodiments of the present invention, is illustrated. In one embodiment, the implantable device 200 comprises a battery unit 210, a power-source controller 220, a stimulation controller 230, a stimulation unit 250, a memory unit 265, and a communications unit 260. The implantable device 200 may also comprise a phasic pulse description array 240, which in one embodiment resides in a memory space (e.g., memory unit 265) in the implantable device 200. The phasic pulse description array 240 comprises data for setting various parameters of the pulses of a stimulation signal, such as current amplitude, pulse-width, frequency, pulse polarity, pulse-shape, and the like. The IMD 200 may also comprise a burst description array 245, comprises data relating to performing a pulse-to-pulse variation of a stimulation signal. The memory unit 265, in one embodiment, is capable of storing various data, such as operation parameter data, status data, and the like, as well as program code.

It will be recognized that one or more of the blocks 210-265 (which may also be referred to as modules) may comprise hardware, firmware, software units, or any combination of the three. The memory unit 265 may be used for storing various program codes, starting data, and the like. The battery unit 210 comprises a power-source battery that may be rechargeable. The battery unit 210 provides power for the operation of the IMD 200, including electronic operations and the stimulation function. The battery unit 210, in one embodiment, may be a lithium/thionyl chloride cell or, in another embodiment, a lithium/carbon monofluoride cell. The terminals of the battery unit 210 may be electrically connected to an input side of the power-source controller 220.

The power-source controller 220 preferably comprises circuitry and a processor for controlling and monitoring the power flow to various electronic and stimulation-delivery portions of the IMD 200. The processor in the power-source controller 220 may be capable of executing program code. In one embodiment, the power-source controller 220 is capable of monitoring the power consumption of the IMD 200 and generating appropriate status signals.

The communication unit 260 is capable of providing transmission and reception of electronic signals to and from an external unit 270. The external unit 270 may be a device that is capable of programming various modules and stimulation parameters of the IMD 200. In one embodiment, the external unit 270 is a computer system that is capable of executing a data-acquisition program. The external unit 270 is preferably controlled by a medical professional, such as a physician, at a base station in, for example, a doctor's office. The external unit 270 may be a computer, in one embodiment, a handheld computer or PDS, but may alternatively comprise any device that is capable of electronic communications and programming. The external unit 270 may download various parameters and program software into the IMD 200 for programming the operation of the implantable device. The external unit 270 may also receive and upload various status conditions and/or other data from the IMD 200. In one embodiment, the external unit 270 may download data relating to the phasic pulse description array 240 and/or data relating to the burst description array 245 for implementation of a multi-phasic stimulation signal. Communications between the external unit 270 and the communication unit 260 may occur via a wireless or other type of communication illustrated generally by a line 275 in FIG. 2.

Stimulation controller 230 defines the stimulation pulses to be delivered to the nerve tissue according to parameters that may be preprogrammed into the IMD 200 using the external unit 270. The stimulation controller 230, which may comprise a processor that can execute program code, controls the operation of the stimulation unit 250, which generates the stimulation pulses according to parameters defined by the pulse description array 240 and provides these pulses to the connector 120 for delivery to the patient via lead assembly 122 and electrode assembly 125. Based upon data from the phasic pulse description array 240, the stimulation unit 250 is capable of implementing multi-phasic controlled current signal outputs. The stimulation unit 250 is capable of providing a controlled current signal where pulses may comprise various amplitudes, varying phases, and varying polarity. The stimulation unit 250 is also capable of providing mono-phasic stimulation signals. The stimulation unit 250 may also be capable of switching between various electrodes employed by the implantable device 200. Programming the IMD 200 for performing multi-phasic stimulation may be provided by the external unit 270 via the communications link 275. The phasic pulse description array 240 may select a particular parameter set defining a multi-phasic pulse pattern, which is then employed by the stimulation unit 250. Various stimulation signals may be provided by the IMD 200.

In an alternative embodiment, based upon various parameters provided by the external unit 270, the stimulation controller 230 may develop a multi-phasic pulse description pattern and provide the same to the stimulation unit 250 to perform a particular type of multi-phasic stimulation. The phasic pulse description array 240 comprises stored description of the phase attributes for the stimulation signal. The stimulation controller 230 is capable of converting the stored data relating to the phasic pulse description array 240 and controls behavior of the stimulation unit 250 accordingly. Additionally, the IMD 200 also comprises a burst description array 245 that comprises data relating to performing a pulse-to-pulse variation of a stimulation signal. The controller 230 is capable of using data from the burst description array 245 to provide a stimulation signal that comprises a pulse train, where one pulse in the pulse train may vary from another pulse train. This pulse-to-pulse variation may include variations in the pulse width, amplitude, pulse-shape, polarity, etc. A more detailed description of the stimulation unit 250 is provided in various figures and accompanying description below.

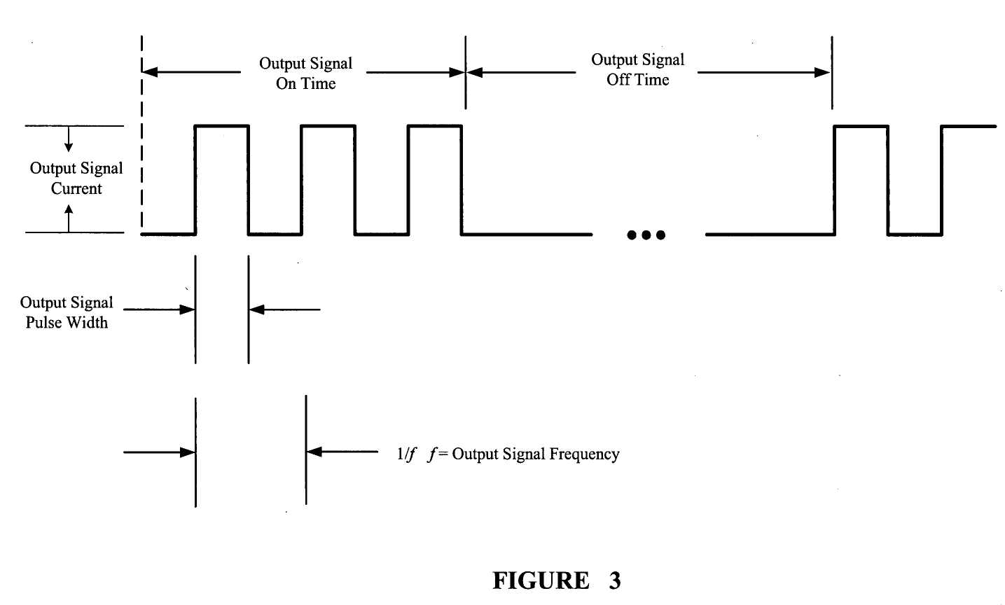

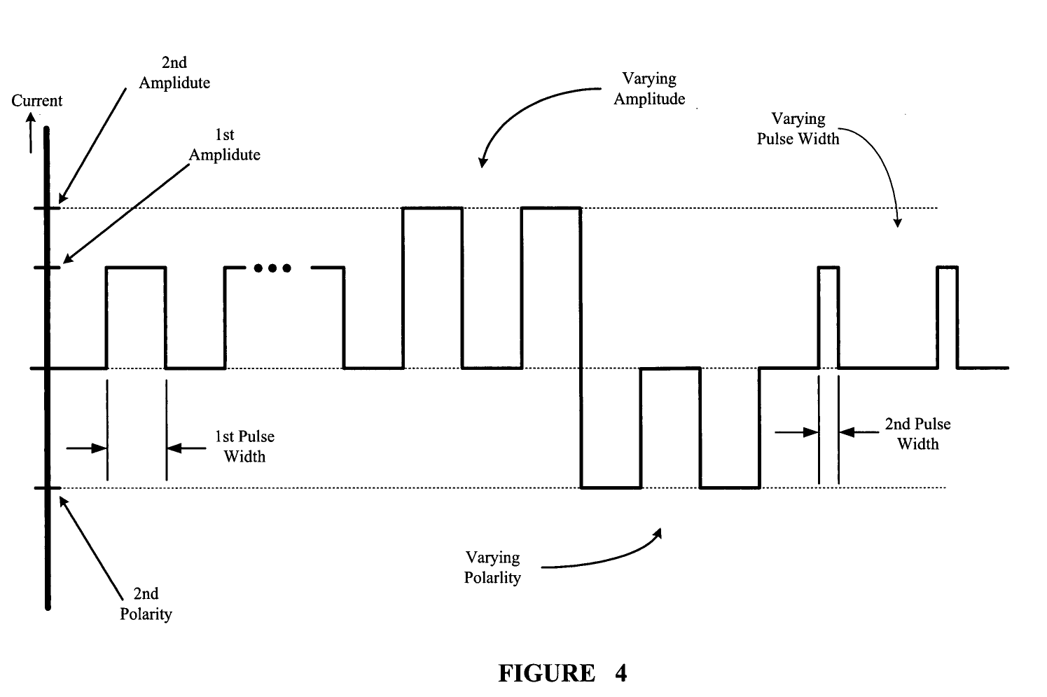

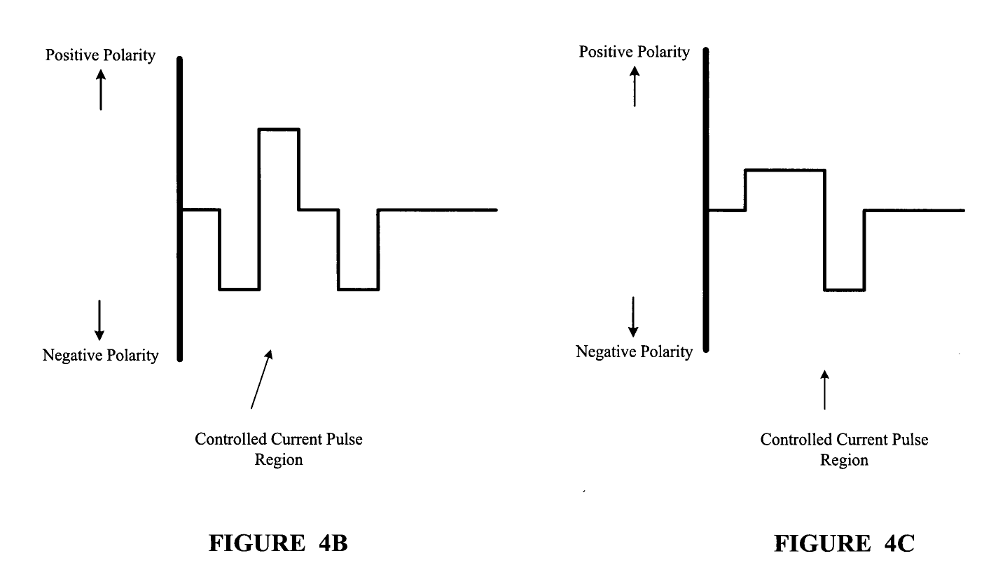

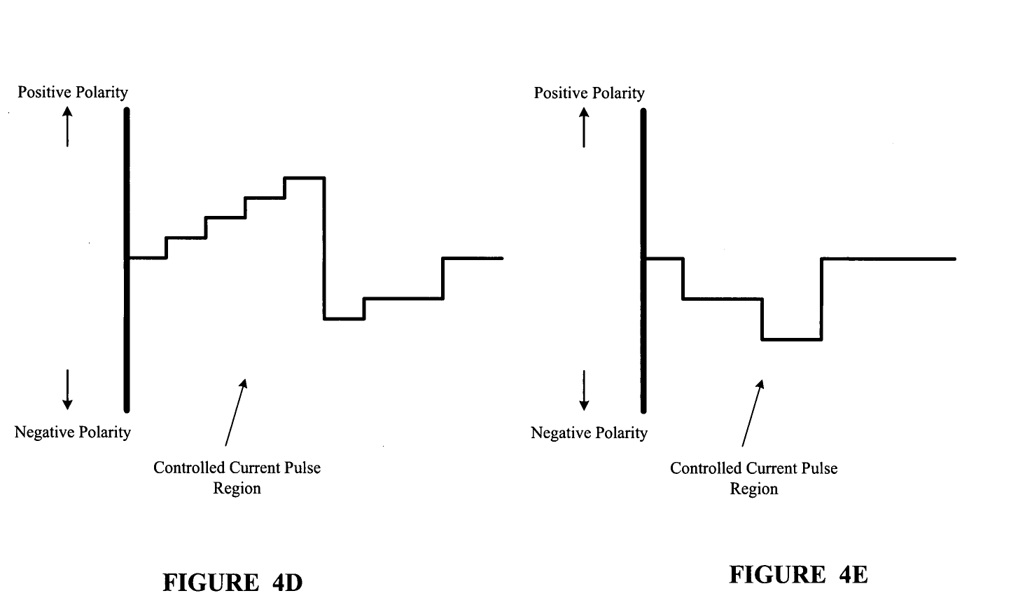

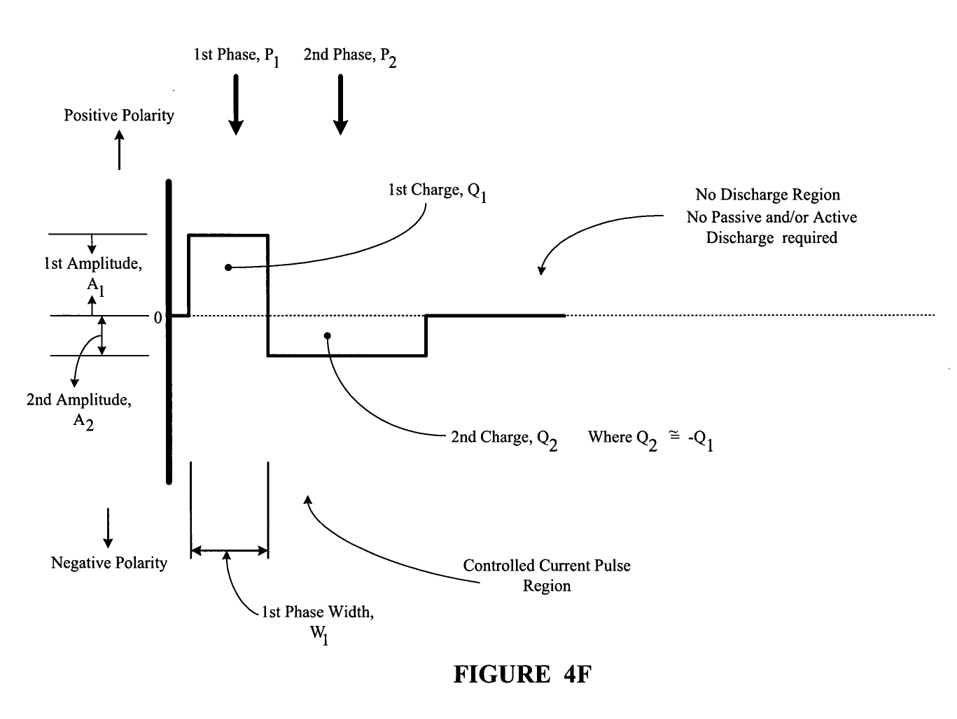

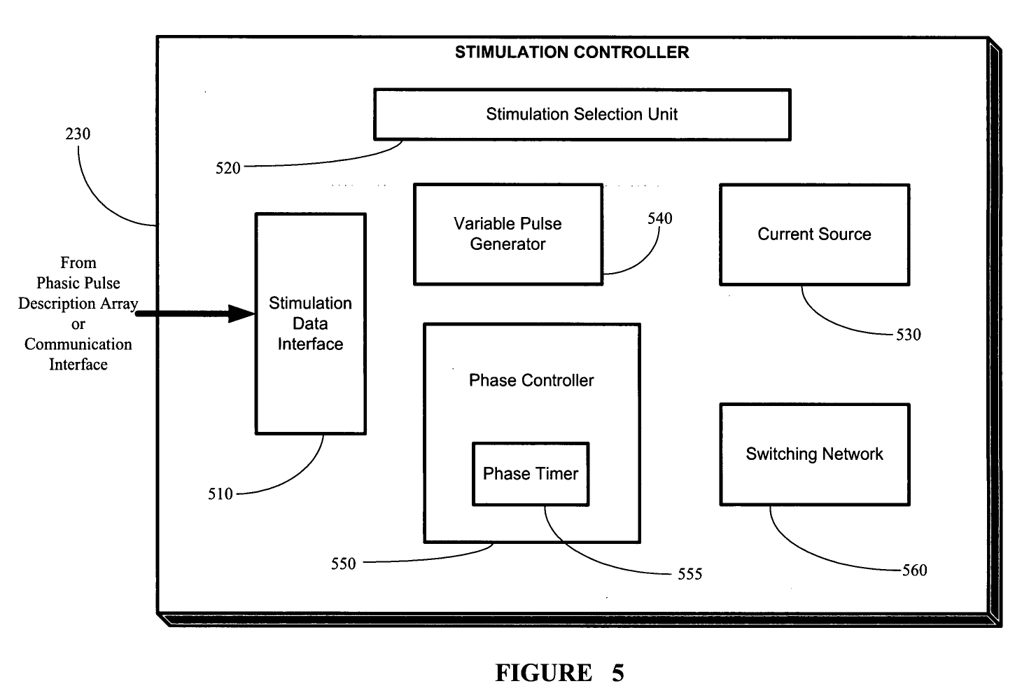

The operation of stimulus generator 110 or the stimulation controller 230 to control and treat the medical conditions of interest is described with reference to FIGS. 3 and 4, which illustrate the general nature, in idealized representation, of output signal waveforms delivered by the output section of the neurostimulator to electrode assembly 125. FIG. 3 illustrates waveforms currently used in prior art IMDs. FIGS. 4A-4F illustrate waveforms suitable for use in embodiments of the present invention. The illustrations are presented principally for the sake of clarifying terminology, including the parameters of output signal on-time, off-time, frequency, pulse width, and current.

FIG. 4A illustrates an exemplary multi-phasic current signal provided by embodiments of the present invention. Many of the stimulation concepts described in the context of FIG. 3 may also apply to multi-phasic signals illustrated in FIG. 4A. Certain parameters may change, however, for particular pulses in a pulse train. In particular, as FIG. 4A illustrates, the pulses of the controlled current signal provided by the IMD 200 may vary in amplitude as illustrated by some pulses having a first amplitude and other pulses having a second amplitude. Furthermore, the polarity of the current signal may vary as indicated by some pulses having a first polarity, indicated by the pulses having a peak above the horizontal zero current line, and other pulses having a second, opposite polarity as indicate by a peak below the zero current line. The signal pulses may also vary in pulse widths as illustrated by the pulses having a first pulse width and a second pulse width, respectively, in FIG. 4A.

The multi-phasic controlled current pulse provided by the IMD 200 may be directed to performing selective activation of various electrodes (described below) and/or to perform targeting particular tissue for excitation. An exemplary multi-phasic stimulation pulse signal provided by the IMD 200 is illustrated in FIG. 4B, where alternating polarity of a pulse signal is illustrated. In one embodiment, the alternating polarity may be employed in conjunction with alternating electrodes for targeting specific tissues. The exemplary stimulation signal illustrated in FIG. 4C depicts a pulse variation in amplitude, pulse width, as well as in polarity. FIG. 4D illustrates an exemplary stimulation signal with a multi-phasic pulse that comprises “stair-step” changes in amplitude, followed by variations in polarity. Therefore, a plurality of phases within a pulse may correspond to a plurality of amplitudes. FIG. 4E illustrates an exemplary stimulation signal with a multi-phasic pulse that provides various phases that correspond to a negative change in amplitude and a change in polarity. As described above, a phase of a pulse may take on various shapes and current levels, including a current level of zero Amps. In one embodiment, a phase with zero current may be used as a time delay between two current delivery phases of a pulse.

FIG. 4F illustrates a multi-phasic pulse signal and has a first phase that corresponds a first amplitude relating to a first charge, Q₁, and a second phase that corresponds to a second amplitude relating to a second charge, Q₂. In the signal illustrated in FIG. 4F, the second charge Q₂ is substantially equal to the negative value of the first charge Q₁. Therefore, the charges, Q₁ and Q₂, balance each other, reducing the need for active and/or passive discharging of the charges. Hence, the pulse signal illustrated in FIG. 4F is a charge-balanced, multi-phasic, controlled current pulse signal. Reducing the need for performing active and/or passive discharge may provide various advantages, such as power savings from the reduction of charge discharge, less circuit requirements, and the like. Various other pulse shapes may be employed in the multi-phasic concepts provided by embodiments of the present invention and remain within the scope and spirit of the present invention.

Turning now to FIG. 5, a more detailed block diagram depiction of the stimulation controller 230 from FIG. 2 is illustrated. The stimulation controller 230 may comprise a stimulation data interface 510 to receive data defining the stimulation pulses, and a stimulation selection unit 520 that is capable of selecting a type of stimulation to be performed by the stimulation controller 230. The stimulation controller 230 is capable of providing a digital control of the pulses provided by the IMD 200. In an alternative embodiment, the stimulation controller 230 is capable of providing an analog control of the pulses provided by the IMD 200.

The stimulation data interface 510 is capable of interfacing with various other portions of the IMD 200. For example, the stimulation data interface 510 may interface with the communication unit 260 (FIG. 2) to receive data from the external unit 270 for determining a particular type of stimulation to be performed. In one embodiment, the stimulation data interface 510 may receive data from the phasic pulse description array 240, which may provide data relating to the type of pulses to be delivered as the stimulation signal. The stimulation data interface 510 may provide data to the stimulation selection unit 520, which then selects a particular type of stimulation to be delivered by the IMD 200.

In one embodiment, the stimulation selection unit 520 may be a hardware unit comprising a processor capable of executing a program code. In an alternative embodiment, the stimulation selection unit 520 may be a software unit, a firmware unit, or a combination of hardware, software, and/or firmware. The stimulation selection unit 520 may receive data from the external unit 270 prompting the unit 520 to select a particular stimulation pulse regime for delivery by the IMD 200. In one embodiment, the stimulation selection unit 520 may receive a phasic pulse description from the phasic pulse description array 240 that describes a particular type of stimulation signal with multi-phasic pulses to be delivered by the IMD 200. In an alternative embodiment, the stimulation selection unit 520 may calculate the type of multi-phasic stimulation pattern to be utilized by the stimulation unit 250 based upon data received from the external unit 270. Therefore, the stimulation data interface 510 receives data relating to the particular type of stimulation signal to use, wherein the stimulation selection unit 520 uses the data from the stimulation data interface 510 to implement the desired stimulation with desired signal characteristics.

A variable pulse generator 540 may generate a varying electrical pulse shape according to the stimulation signal defined by the stimulation selection unit 520. Based upon the data relating to the type of stimulation to be delivered, the stimulation selection unit 520 provides control signals for selecting a particular type of stimulation signal to be delivered by the IMD 200. The variable pulse generator 540 is capable of generating a number of electrical pulse waveforms for use as the stimulation signal. The pulses may comprise various shapes such as a square wave, a triangular wave, a stepped leading edge and/or trailing edge type pulse, and other pulse shapes. Moreover, a plurality of such shapes may be specified within a single pulse train and/or in sequential pulse trains. Particular shapes may be used for various reasons, such as targeting particular nerve fibers, performing pre-polarization, or hyper-polarization, and the like. The variable pulse generator 540 preferably comprises timing devices and other electronic circuitry for generating the signal pulses.

The stimulation controller 230 also comprises a current source 530 to provide a controlled current signal for delivery of stimulation pulses to the patient. The current source 530, in one embodiment, is capable of providing a controlled current even if the impedance across the leads varies (as described below), thereby delivering the stimulation signal from the implantable device 200 to a target portion of the patient's body. A more detailed description of the current source 530 is provided in FIG. 7 and the accompanying description below.

Referring again to FIG. 5, the stimulation controller 230 may also comprise a phase controller 550 for controlling various phases of the stimulation signal. For example, the phase controller 550 may determine the “on” time and the “off” time of each of the pulse phases to be controlled by the stimulation controller 230. The stimulation controller 230 performs the action as defined by either the phasic pulse description array 240 and/or the burst description array 245. The phase controller 550 provides a first phase control signal prompting the stimulation controller 230 to begin delivering a first type(s) of pulses to the patient. The type of pulse may include various mono-phasic and/or multi-phasic pulses with various shapes, such as the exemplary mono-phasic and/or multi-phasic pulses illustrated in FIGS. 3 and 4A-4F. The phase controller 550 may thereafter provide a second phase control signal to terminate delivery of the first type of pulses and begin delivery of a second type of pulses to the patient. The first and second phase control signals may be delivered during a single pulse train or between separate pulse trains. The phase controller 550 may also comprise a phase timer 555, which provides timing control for marking the beginning and end of particular portions of a multi-phasic signal provided by the IMD 200. The phase timer 555 may be any type of timer that is capable of providing timing signals to enable the phase controller 550 to begin and end various phases.

Additionally, the stimulation controller 230 may comprise a switching network 560 capable of switching through various polarities and wires. For example, the switching network 560 may switch between various electrodes that may be driven by the IMD 200. Additionally, the switching network 560 may provide a switching mechanism for performing pulse control, as directed by the phase controller 550, to control the pulses provided by the IMD 200. The pulse control may include controlling the various shapes of the pulses, during the duration of the pulse, thereby providing a multi-phasic and/or a non-phasic pulse signal. Thus, using particular sub-modules of the stimulation controller 230 (e.g., sub-modules 510-560), the implantable device 200 is able to deliver various pulses in various shapes, durations, and polarities, and deliver the stimulation signal to multiple electrodes in various combinations.

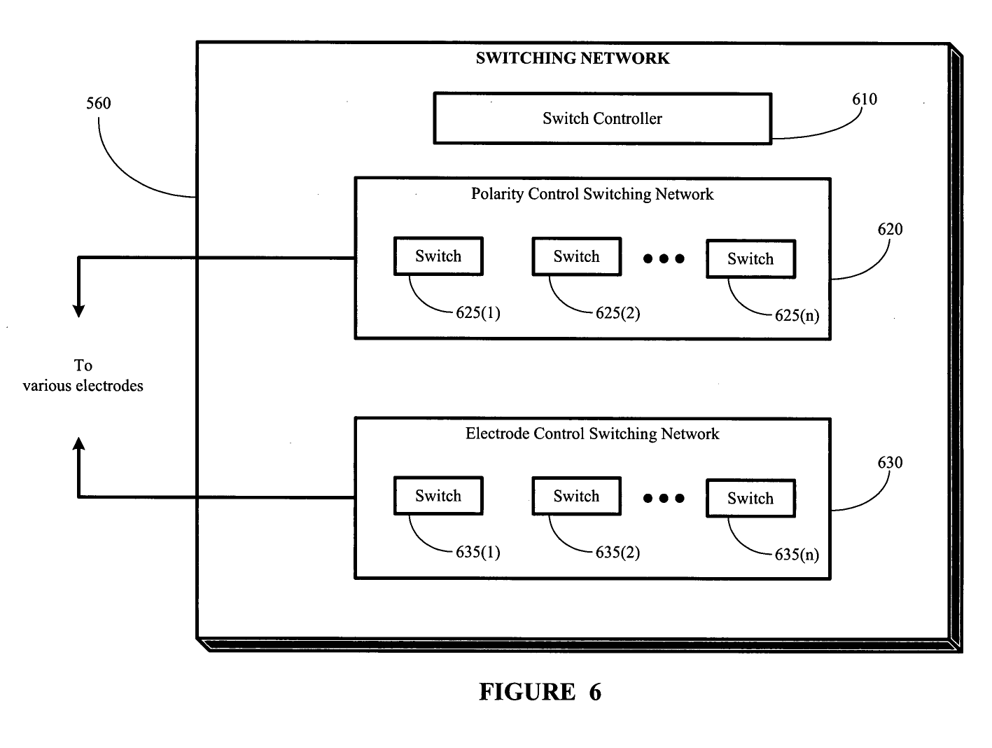

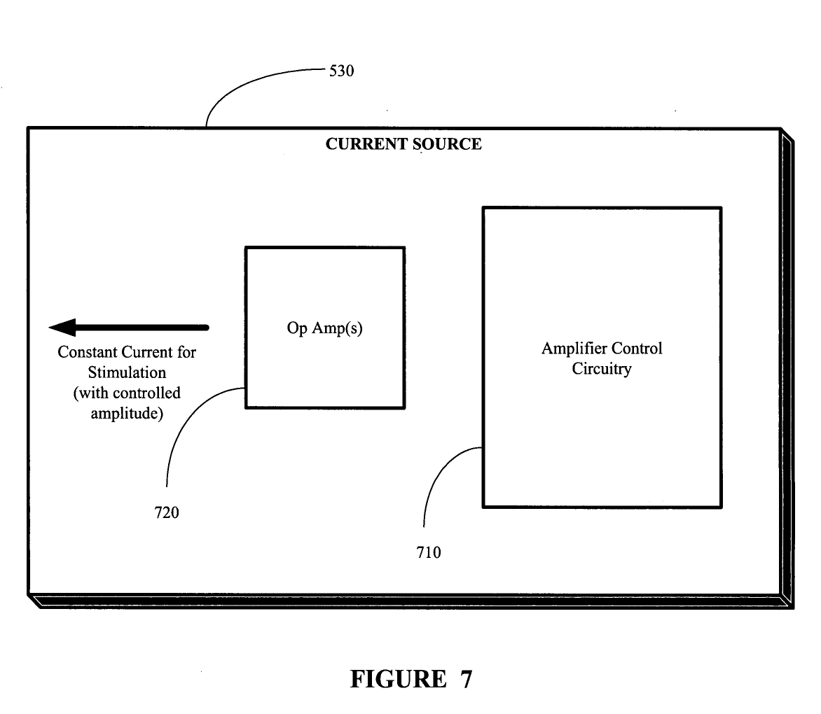

FIG. 6 provides a block diagram depiction of the switching network 560 (FIG. 5) in accordance with embodiments of the present invention. In one embodiment, the switching network 560 may comprise a switch controller 610 for controlling a plurality of switches in the switching network 560. The switching network 560 may receive data from the various sub-modules of the stimulation controller 230 (e.g., 520-555 of FIG. 5), and activate various switches to perform pulse control of the stimulation signal and/or select various combinations of electrodes used by the IMD 200 to deliver stimulation. The switching network 560 preferably comprises a polarity control switching network 620 for controlling the polarity of a selected electrode (i.e., whether it will be used as a cathode or anode) and an electrode selection switching network 630 for selecting which electrodes among a plurality of electrodes are to be used to deliver stimulation pulses. Both polarity control switching network 620 and electrode selection switching network 630 may be controlled by the switch controller 610.

In one embodiment, the polarity control switching network 620 may comprise a plurality of switches 625(1)-625(n). The switches 625(1)-625(n) may be electro-mechanical switches, solid state switches, transistors, and/or any other types of switches. In a preferred embodiment, the switches comprise transistors. The polarity control switching network 620 may comprise switches 625 that are arranged in such a fashion that control signal(s) may toggle particular switches 625 to effect a reversal of the polarity of a pair of wires associated with a particular lead assembly. Thus, the polarity control switching network 620 is capable of reversing the polarity of a plurality of electrodes coupled to one or more target portions of the patient's body. The changing of the polarity of one or more pulses of a stimulation signal may be performed in conjunction with the change in a particular phase of the stimulation signal.

The electrode control switching network 630 may comprise a plurality of switches 635(1)-635(n) capable of switching so as to allow selected electrodes to be activated by the IMD 200. The switches 635(1)-635(n) may be electro-mechanical switches, solid state switches, transistors, and/or any other types of switches. In a preferred embodiment, the switches comprise transistors. The signal to be directed by the stimulation unit 250 is guided by the activation or deactivation of various switches 635 such that predetermined electrode pairs from among a plurality of electrode pairs are selected to deliver the stimulation signal to one or more target locations of the patient's body. Based upon information from the stimulation selection unit 520 in the stimulation controller 230, the switch controller 610 directs stimulation signals to predetermined electrodes by using the electrode control switching network 630 for targeting particular areas of the patient's body. Likewise, the polarity of these electrodes may be defined based upon information from the phasic pulse description array 240, which may call for reversing the polarity of various pulses within a pulse train, which is controlled by the activation or deactivation of the switches 625(1-n) controlled by the switch controller 610. Thus, the switching network 560 is utilized by the stimulation controller 230 to perform polarity control and selection of various combinations of electrodes connected to the implantable device 200.

FIG. 7 illustrates one embodiment of a current source 530 (FIG. 5) in the stimulation controller 230 of FIG. 2. The current source 530 may comprise an op amp block 720, which in turn may comprise one or more operational amplifiers (not shown) that are capable of delivering a controlled current signal for therapy or stimulation. The current source 530 may also comprise amplifier control circuitry 710 that with circuitry and/or programmable logic to control the operation of the op amps 720.

Embodiments of the present invention provide for utilizing the delivery of a controlled current signal for stimulation via leads and electrodes. In one embodiment, the controlled current signal provided by the current source 530 is independent of the impedance experienced across the leads. Thus, even if the impedance experienced by the leads changes, the op amp 720 in the current source 530 is preferably designed, in conjunction with the amplifier control circuitry 710, to adjust and continue to deliver a controlled current stimulation despite variations in the lead impedance. For example, if the nerve tissue to which the electrodes are coupled has an impedance of 1000 ohms, and a particular stimulation therapy requires a one milliamp controlled current signal, the current source 530 will still provide the required one milliamp current even if the nerve tissue (and/or the lead wires themselves) change so as to provide a 5000 ohms impedance across the leads. Hence, while the power may vary, the current remains constant. In other words, the op amp 720 will stabilize itself utilizing circuitry, including the amplifier control circuitry 710, to provide a constant one milliamp current signal even if the impedance experienced by the leads changes.

The current source 530 is capable of delivering a controlled current signal with various amplitudes. In other words, the amplitude of the current generated for stimulation is controlled by the current source 530. As described above, data from the phasic pulse description array 240 defines the pulses of the stimulation signal, more specifically; the array 240 defines the phases of the multi-phasic pulses. The data from the burst description array 245 defines the pulse train signals. Data from the stimulation selection unit 520 may be used by the current source 530 to adjust the output of the op amps 720 such that current pulses of various amplitudes at desired times or desired phases in a stimulation pulse train are provided for implementation of the multi-phasic stimulation provided by embodiments of the present invention. More specifically, in one embodiment, the amplifier control circuitry 710 comprises circuit(s) capable of receiving control signals from the stimulation selection unit 520, which may be based upon data from the phasic pulse description array 240 and/or the burst description array 245, to control the output of the op amp 720 to provide a desired amplitude. The current source 530 provides a predetermined current output that is independent of varying impedances seen by the IMD 200 and may provide variable amplitudes for targeting performing various types of stimulation.

The term “controlled current” as used herein does not refer to a pulse signal having a constant or uniform current amplitude for all pulses in a pulse train (or even within a single pulse). Instead, it refers to a current whose magnitude is independent of the lead impedance, and the amplitude of the pulses is as defined by the stimulation selection unit 520. This definition of the stimulation signal by the stimulation selection unit 520, in one embodiment, is based upon data from the phasic pulse description array 240 and/or the burst description array 245. Indeed, as shown in FIGS. 4A-4F and as described above, the current source 530 may provide a first pulse with a first current amplitude that is uniform throughout the pulse, and a second pulse with a second current amplitude, different from the first amplitude, also uniform throughout the pulse. Certain pulses, e.g., a stepped leading or trailing edge pulse, or a triangular shaped pulse, require a varying current even within the pulse itself. All such pulses are “controlled current” pulses within the meaning of the present invention so long as the current amplitude is independent of the lead (or lead/electrode) impedance.

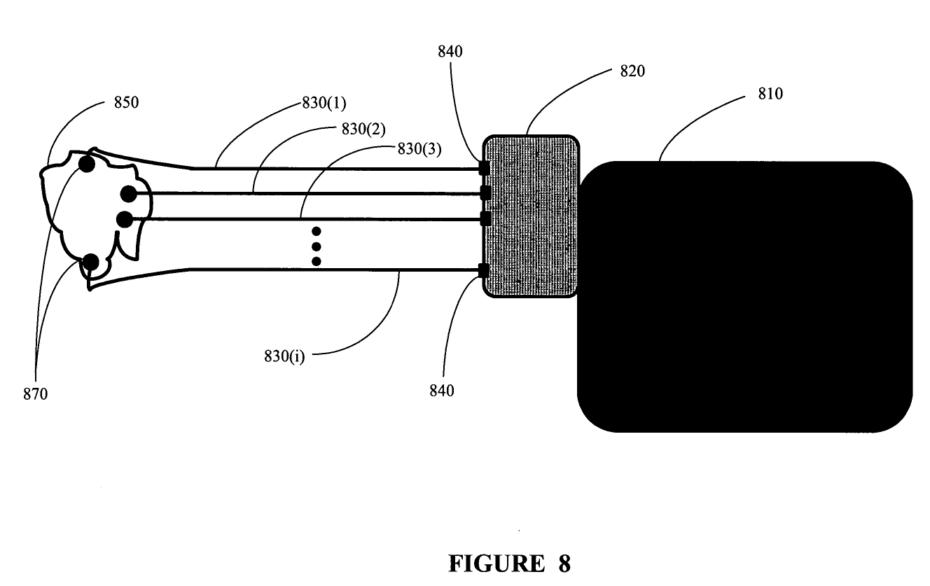

Turning now to FIG. 8, a stylized illustration of an implementation of the IMD 200 is illustrated. The IMD 200 may comprise a main body 810 in which the electronics described in FIG. 2 are enclosed and hermetically sealed. Coupled to the main body 810 is a header 820 designed with terminal connectors 840 for connecting to the various leads 830(1) through 830(i). The main body 810 may comprise a titanium shell, and the header 820 may comprise a clear acrylic or other hard, biocompatible polymer such as polycarbonate, or any material that may be implantable into a human body. The leads 830 projecting from the header 820 may be attached to a target portion of tissue 850 utilizing a variety of methods for attaching the lead 830 to the tissue 850. The target portion of the patient's body 850 may comprise any of a number of locations within a patient, such as a nerve bundle, an area of nerve fiber, an area of a muscle, bone or organ tissue, and the like. A first end of the leads 830 is coupled to connectors 840 on the header 820, which are electrically coupled to the main body 810 via the header 820 to the tissue 850. Therefore, the current flow may take place from one terminal of the lead 830 to a second terminal of the lead 830 via the tissue 850, thereby delivering the stimulation. A plurality of second ends of the leads 830 are electrically coupled to a plurality of electrodes 870, which are electrically and/or mechanically coupled to the tissue 850.

The system illustrated in FIG. 8 may be viewed as an electrical circuit that includes a current or voltage source (i.e., the IMD 200) being connected to an impedance (i.e., the equivalent impedance of the tissue 850) via a pair of wires (i.e., the leads 830). The total impedance connected to the IMD 200 includes the impedance of the wires as well as the impedance across the terminals of the leads 830 to the tissue 850. One of the biggest components of the impedance experienced by terminal connectors 840 on the header 820, to which the leads 830 are connected, is the impedance of the tissue 850.

As illustrated in FIG. 8, the IMD 200 is preferably capable of driving a plurality of leads 830(1)-830(i), which are connected to various electrodes 870 on the tissue 850. In one embodiment, the implantable device 200 is capable of driving stimulation signals on one or more of the leads 830(1-i) to activate various electrodes 870. The switching network 560 described above is capable of selecting various combinations of electrodes 870 to be activated by switching on or off the current signal onto different combinations of the leads 830(1-i). Utilizing the embodiments illustrated in FIG. 8, multi-phasic stimulation may be provided with the added benefit of selective activation of multiple electrodes 870.

In one embodiment, the configuration of the IMD 200 and electrodes 830(1-i) illustrated in FIG. 8 may be used to provide a stimulation signal to a target portion of a patient's body to provide a sequential stimulation at approximately 90 degree angles. In order to perform this type of stimulation, the electrodes 830(1-i) may be coupled to an area associated with the target portion of the patient's body in an orthogonal configuration. In a alternative embodiment, the configuration of the IMD 200 and electrodes 830(1-i) illustrated in FIG. 8 may be used to provide a stimulation signal to a target portion of a patient's body to provide a sequential stimulation in a round robin fashion. In yet another alternative embodiment, the plurality of electrodes 830(1-i) may be coupled to a plurality of target portions (e.g., a 1^{st} through ith nerve) of the patient's body.

Utilizing the multi-phasic techniques and selective combination of electrodes 870 described above, hyper-polarization prior to de-polarization may be performed to allow for selective stimulation of fibers, nerve fibers, and/or other portions of a patient's body. Pulse parameters with varying amplitudes, durations, polarities, and/or various shapes, in conjunction with selective electrodes may be chosen to hyperpolarize, depolarize, and/or repolarize, various portions of the patient's body to increase neural conduction or neural inhibition. Utilizing these techniques increases efficacy and/or provides for reducing complications via selective activation or selective inhibition. This provides afferent selectivity, vocal stimulation prevention, Brady/syncope prevention (i.e., preventing efferent stimulation that may impact cardiac function), and/or the like. Furthermore, the dynamic polarity alteration provided by embodiments of the present invention provides for post implant reversal of polarity of selected leads/electrodes to reduce complications that may occur in a patient's body.

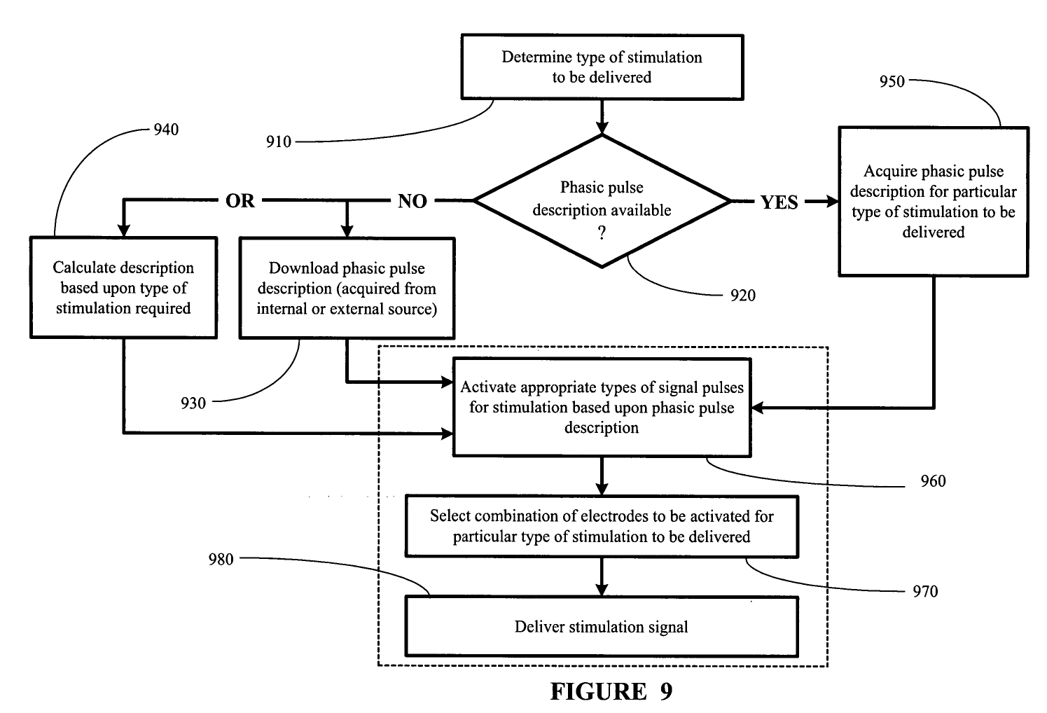

Turning now to FIG. 9, a flow chart depiction of the steps for performing multi-phasic stimulation according to embodiments of the present invention is illustrated. A determination is made regarding the type of stimulation that is desired for a particular patient (block 910). In one embodiment, these determinations may be made prior to the implantation of the IMD 200 and/or may be determined by a medical professional, e.g., a physician, at a later time. The external unit 270 may be used to program the IMD 200 to change one or more parameters of the stimulation, which may change or replace the multi-phasic pulse description array stored in the IMD 200. Alternatively, a library of defining a plurality of stimulation regimes may be provided in a memory of the IMD 200 and may be accessed by the physician for implementation. In another alternative embodiment, the library may be automatically accessed and a particular stimulation regime implemented as a result of sensor inputs received by the IMD 200 from sensors located in the patient's body.

Following the determination of which type of stimulation is desired, the IMD 200 may make a determination whether the phasic pulse description, based upon the particular stimulation that is to be implemented, is immediately available (block 920). If a determination is made that the particular phasic pulse description is not immediately available, in one embodiment, the IMD may take at least one of two actions. First, the IMD 200 may download the phasic pulse description, which may be from an internal source, e.g. data stored in the phasic pulse description array 240 and/or the burst description array 245, or in the memory unit 265 (block 930). In an alternative embodiment, the phasic pulse description may be downloaded from the external device 270.

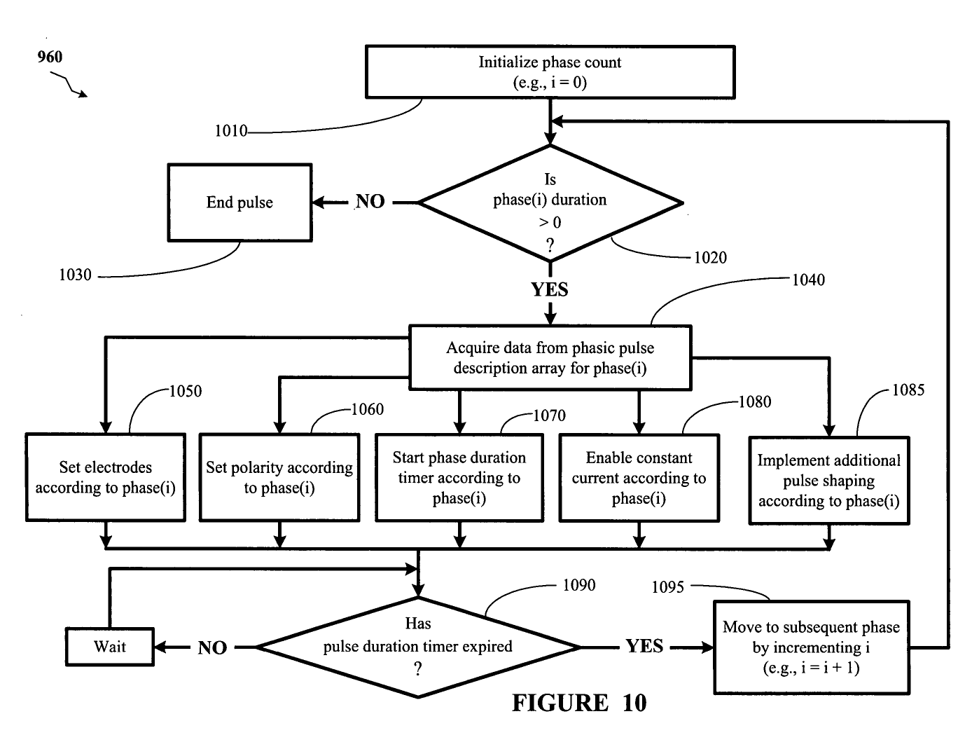

Alternatively, the IMD 200 may itself calculate the phasic pulse description based upon the type of stimulation desired (block 940). In one embodiment, the stimulation controller 230 is capable of calculating the various pulse descriptions, i.e., the current amplitude, the pulse shape, the phase(s), the polarity, and/or the combination of electrodes that are to be implemented in the delivery of the pulse train desired. If the phasic pulse description is available, the stimulation controller 230 of the IMD 200 may acquire the phasic pulse description for preparation of controlling the stimulation unit 250 for generation and delivery of the multi-phasic signal (block 950). The IMD 200 may then activate the desired pulses as called for by the phasic pulse description to create a stimulation signal in accordance with the phasic pulse description (block 960). In order to deliver a multi-phasic stimulation signal, the phasic pulse description may be calculated, downloaded, or received from the phasic pulse description array 240. In order to deliver a pulse train stimulation signal, which may include one or more multi-phasic pulses, the signal description may be calculated, downloaded, or received from the burst description array 245 and/or the phasic pulse description array 240. A more detailed description of implementing the activation of the multi-phasic stimulation signal is provided in FIG. 10 and the accompanying description below.

After the controller 230 acquires the information to characterize the type of stimulation signal to be implemented based upon the phasic pulse description, the actual stimulation is then delivered to target portions of the patient's body by the IMD 200. This includes selecting a particular combination of electrodes 870 for activation by the switching network 560 to deliver stimulation signals to the target portion(s) of the patient's body (block 970). The switching network 560 may be utilized to actuate various switches 635 such that selected electrodes 870 coupled to the target portions of the patient's body are chosen for delivery of the multi-phasic and/or single-phasic phase stimulation signal. Based upon the appropriate type of stimulation signal to be activated, and the selected combination of electrodes, the stimulation signal is provided for delivery of stimulation therapy block (980). The stimulation signal (i.e., pulse train) may comprise a plurality of phases, wherein a particular characteristic of the signal (e.g., pulse-width, pulse-amplitude, pulse-polarity, pulse-shape, etc.) may be varied for some or all pulses in the pulse train. For example, in a first time period of a pulse train, the pulse width and the pulse amplitude may be varied for selected pulses, while during a second time period, the polarity of the selected pulses may be varied.

Turning now to FIG. 10, a more detailed flow chart depiction is provided of the step (960 in FIG. 9) of activating the various types of stimulation characterized by the phasic pulse description, in accordance with embodiments of the present invention. Although, the description provided by FIG. 10 is presented in the context of delivering a multi-phasic pulse stimulation signal, concepts provided in FIG. 10 may also be used to deliver a pulse train stimulation signal. When performing a multi-phase signal stimulation, a phase count is initialized to begin the multi-phase stimulation signal delivery. In one embodiment, the phase count is initialized to zero (e.g., i=0) (block 1010). Once a phase count relating to a particular phasic pulse description is initialized, a determination is made whether the remaining duration of a particular phase (i.e., phase(i)) is greater than zero (block 1020). When it is determined that the particular phase duration is not greater than zero, then the pulse (i.e., a multi-phasic pulse) is ended (block 1030).

If it is determined that the remaining phase duration of phase(i) is greater than zero, then data from the phasic pulse description array 240 characterizing that particular phase (phase(i)) is acquired (block 1040). In one embodiment, the stimulation selection unit 520 in the stimulation controller 230 acquires the data characterizing the stimulation signal in accordance with the phasic pulse description. Upon acquisition of the phasic pulse description for the particular phase of a multi-phase signal, or in the case of a pulse train signal, a burst description, the electrodes 870, according to the particular phase, phase(i), are selected for activation (block 1050). For example, the electrodes 870 that relate to leads 830(1), 830(2), and/or other leads up to lead 830(i) are selected by the activation of various switches in the switching network 560. Therefore, targeted stimulation delivery to particular target portions of the patient's body is provided. Additionally, the acquisition of the phasic pulse description data also characterizes the polarity of each portion of the pulse associated with a particular phase according to the requirements of the phase, (block 1060). Therefore, in one embodiment, the polarities of pulses according to the phase(i) description may be selected by the switching network 560 to set the polarity of the pulses comprising the stimulation signal.

Also based upon the phasic pulse description, a phase duration timer for characterizing a phase width is started according to the description in relation to phase(i) (block 1070). In one embodiment, the phase controller 550, which comprises the phase timer 555, initiates the timer and controls the duration of the phase of the pulse. In the case of a pulse train stimulation signal, the timer may be initiated to control the duration of various pulses in the pulse train. When the phase timer 555 indicates that the phase should be ended, it cuts off a particular phase. So, the phase(i) continues as long as the phase timer 555 allows for the continuation of the phase.

The phasic pulse description may also be used to enable a controlled current signal according to requirements of the phase(i) (block 1080). The current source 530 is controlled to provide a controlled current with particular amplitude(s), according to the phasic pulse description for phase(i). Furthermore, the phasic pulse description array 240 may provide data for further shaping a particular pulse during a phase(i) (block 1085). The variable pulse generator 540 may generate a pulse of varying shapes, such as a ramp-up shape, a square pulse, and/or the like, in accordance with the pulse description for the particular phase(i). In one embodiment, a stimulation defined by parameters associated with any combination of the signal control blocks 1050, 1060, 1070, 1080 and/or 1085, may be implemented. The processes associated with blocks 1050, 1060, 1070, 1080 and/or 1085 may be performed sequentially or in parallel. The implementation of a change in a stimulation parameter associated with one or more of the control blocks may or may not be called for by a particular pulse description, and if not the stimulation may fall back to a default stimulation (in terms of current amplitude, electrodes used, pulse polarity, duty cycle, frequency, and other parameters). A default configuration for the pulse shape, the duration of the phase, the polarity of the phase, and for the particular electrode selected may be predetermined for utilization by the IMD 200.

The shape of the pulse may determine the total duration of the pulse, which may be provided by the data described in block 1085. In the case of a pulse train signal, data relating to block 1085 may provide the pulse width relating to the pulses in the pulse train. After the type of multi-phasic pulse signal to be delivered has been defined, a determination is made whether the duration of the phase timer 555 has expired (block 1090). If the duration of the pulse timer 555 for phase(i) has not expired, the IMD 200 may simply wait until the phase timer 555 has expired for further action and continue the delivery of particular types of signals called for by the phasic pulse description. When the pulse timer 555 has expired, the implantable device 200 moves to the next phase by incrementing (i) by a predetermined number, such as incrementing (i) by 1. The next phase is then implemented and the blocks described in FIG. 10 are repeated as indicated in FIG. 10. Although, the flowchart of FIG. 10 provides for delivering a multi-phasic pulse signal, similar flow provided by the flowchart in FIG. 10 may be utilized for providing a pulse train signal, using data from the burst description array 245.

Utilizing embodiments of the present invention a multi-phasic stimulation may be performed characterized by a pulse train in which one or more pulses may differ from other pulses in the train in one or more stimulation parameters. Multi-phasic stimulation in accordance with embodiments of the invention allows a wide variety in the stimulation therapy that may be performed. The IMD 200 described herein is also capable of performing a mono-phase stimulation described above. Implantable medical devices according to the present invention are also capable of selecting a desired combination of electrodes that may be used to stimulation target portions of the patient's body. Utilizing embodiments of the present invention, stimulation of targeted portions of the patient's body, such as nerve fibers, may be realized with more effective stimulation results.

Embodiments of the present invention can be used to hyper-polarize a target nerve prior to de-polarization, or re-polarization. These types of stimulation may provide for increased neural conduction of a stimulation controlled current signal and/or neural inhibition, among various other advantages. Additionally, utilizing embodiments of the present invention, a reduction in the total energy necessary to achieve therapeutic efficacy may be realized. Also, increased efficacy of stimulation therapy may be realized. In other embodiments, post-implant reversal of the polarity of selected leads and/or electrodes may be used to reduce complications by avoiding surgery to change the location of the electrodes. Additionally, wave-shape selection may be used to increase efficacy and/or reduce complications via selective activation and/or selective inhibition of stimulation of a target nerve fiber.

Utilizing embodiments of the present invention, various pulse widths may be used to target particular types of nerve fibers. In addition, by providing pulses of different pulse widths and/or current amplitudes within a pulse train, selective nerve fibers may be targeted for stimulation at different rates. For example, half of the stimulation pulses may be provided so as to stimulate A and B fibers only, while the remaining half of the stimulation pulses in the train may provide stimulation of A, B and C fibers. Thus, the invention allows stimulation of particular fibers more than, or less than, other fibers. Utilizing embodiments of the present invention, a more robust and flexible stimulation of targeted portions of a patient's body may be achieved.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. A method for treating a patient with a multi-phasic stimulation signal from an implantable medical device (IMD), comprising:
applying an electrical pulse with a first controlled characteristic selected from the group consisting of a pulse width, a pulse amplitude, a pulse polarity, and a pulse shape, to a target portion of a vagus nerve using a pulse generator in a first phase defined by a first time period;
modifying said first controlled characteristic of said electrical pulse to provide a second controlled characteristic for said electrical pulse in a second phase defined by a second time period; and
applying said electrical pulse with said second controlled characteristic to said target portion of said vagus nerve.

2. The method of claim 1, wherein modifying said first controlled characteristic of said electrical pulse to provide a second controlled characteristic for said electrical pulse comprises changing at least one of said first amplitude, said first polarity, said first pulse width and said first pulse shape.

3. The method of claim 1, wherein modifying said first controlled characteristic of said electrical pulse further comprises acquiring a phasic pulse description.

4. The method of claim 1, further comprising:
modifying said second controlled characteristic of said electrical pulse to provide a third controlled characteristic for said electrical pulse in a third phase defined by a third time period; and
applying said electrical pulse with said third controlled characteristic to said target portion of said vagus nerve.

5. The method of claim 1, wherein modifying said first controlled characteristic further comprises initializing a second phase.

6. The method of claim 5, wherein initializing said second phase further comprises starting a phase timer and performing at least one of setting a pulse polarity, setting an amplitude, setting a pulse shape, setting a pulse duration and setting a combination of electrodes for said second phase.

7. The method of claim 6, further comprising terminating said second phase upon a time-expiration for said phase timer.

8. The method of claim 1, wherein applying said electrical pulse further comprises applying a pulse train signal to said vagus nerve, wherein at least two pulses in said train comprise controlled multi-phasic pulses.

9. A method for treating a patient with a multi-phasic stimulation signal from an implantable medical device (IMD), comprising:
providing an electrical pulse with a first controlled characteristic selected from the group consisting of a pulse width, a pulse amplitude, a pulse polarity, and a pulse shape;
applying said electrical pulse to a target portion of a vagus nerve during a first phase defined by a first time period;
changing said first controlled characteristic of the electrical pulse during a second phase defined by a second time period to provide a second controlled characteristic for said electrical pulse during a second time period; and
applying said electrical pulse with said second controlled characteristic during said second phase to said target portion of said vagus nerve.

10. An implantable medical device (IMD) for delivering a multi-phasic stimulation signal to a patient, comprising:
a stimulation unit to provide an electrical pulse during a first phase associated with a first time period to a target portion of a vagus nerve, said electrical pulse having a first controlled characteristic during said first time period, said first controlled characteristic comprising at least one of a first amplitude, a first polarity, a first pulse width, and a first pulse shape; and
a controller operatively coupled to said stimulation unit, said controller being adapted to direct said stimulation unit to apply said pulse having said first controlled characteristic to said target portion of the vagus nerve, the controller also being adapted to perform a controlled modification of said first controlled characteristic to generate a pulse with a second controlled characteristic during a second phase associated with a second time period, said second controlled characteristic comprising at least one of a second amplitude, a second polarity, a second pulse width, and a second pulse shape.

11. The implantable medical device of claim 10, wherein said stimulation unit further comprises a phase controller for providing a timing reference for the duration of said first and said second phases, said phase controller comprising a phase timer for providing said timing.

12. The implantable medical device of claim 11, further comprising a phasic pulse description array comprising data for providing at least one controlled characteristic for said signal during said first phase and for providing at least one controlled characteristic for said pulse during said second phase.

13. The implantable medical device of claim 12, further comprising a burst description array comprising data relating to a pulse train stimulation signal.

14. The implantable medical device of claim 10, wherein said controller is further adapted to modify said second controlled characteristic to provide a third controlled characteristic of said pulse during a third phase associated with a third time period, said third characteristic comprising at least one of a third amplitude, a third polarity, a third pulse width, and a third pulse shape.

15. The implantable medical device of claim 10, wherein said stimulation unit is adapted to hyper-polarize said target portion of said vagus nerve using said multi-phasic signal.

16. The implantable medical device of claim 10, wherein said stimulation unit is adapted to pre-polarize said target portion of said vagus nerve using said multi-phasic signal.

17. The implantable medical device of claim 10, further comprising a communication unit to provide communications with an external device.

18. The implantable medical device of claim 10, wherein said stimulation unit is capable of directing said electrical pulse to one or more electrodes associated with said implantable medical device.

19. The implantable medical device of claim 10, wherein said stimulation unit comprises circuitry adapted to perform an active discharge of charges associated with said electrical pulse.

20. The implantable medical device of claim 10, wherein said stimulation unit comprises circuitry adapted to perform a passive discharge of charges associated with said electrical pulse.

21. The implantable medical device claim of 10, wherein said first controlled characteristic comprises a first amplitude, a first polarity and a first pulse width, and said second controlled characteristic comprises a second amplitude, a second polarity and a second pulse width.

22. A computer readable program storage device encoded with instructions that, when executed by a computer, performs a method for treating a patient with a multi-phasic stimulation signal from an implantable medical device (IMD), comprising:
applying an electrical pulse with a first characteristic comprising at least one of a pulse width, a pulse amplitude, a pulse polarity, and a pulse shape, to a target portion of a vagus nerve using a pulse generator in a first time period;
performing a controlled modification of said first characteristic of said electrical pulse to provide a second characteristic for said electrical pulse during a second time period; and
applying said electrical pulse with said second characteristic to said target portion of said vagus nerve.

23. A method for treating a patient with a tri-phasic stimulation signal from an implantable medical device (IMD), comprising:
applying an electrical pulse to a target portion of a cranial nerve using a pulse generator, said electrical pulse comprising a first phase having a first characteristic, a second phase having a second characteristic, and a third phase having a third characteristic, wherein said first characteristic comprises at least one of a first amplitude, a first polarity, a first pulse width, and a first pulse shape, wherein said second characteristic comprises at least one of a second amplitude, a second polarity, a second pulse width, and a second pulse shape, and wherein said third characteristic comprises at least one of a third amplitude, a third polarity, a third pulse width, and a third pulse shape.

24. The method of claim 23, wherein said first characteristic comprises a first polarity, said second characteristic comprises a second polarity opposite said first polarity and said third characteristic comprises a third polarity opposite said second polarity.

25. The method of claim 23, wherein applying said electrical pulse further comprises digitally controlling said first characteristic.

26. The method of claim 25, wherein applying said electrical pulse further comprises digitally controlling said first, second and third characteristics.

27. The method of claim 23, wherein applying said electrical pulse during said third phase further comprises applying a charge-balance signal for balancing an electrical charge resulting from the electrical pulse from at least one of said first phase and said second phase.

28. The method of claim 23, wherein said cranial nerve comprises a vagus nerve.

29. The method of claim 23, wherein applying said electrical pulse to said target portion of said cranial nerve further comprises applying a controlled current pulse.

30. An implantable medical device (IMD) for delivering a multi-phasic stimulation signal to a patient, comprising:
a stimulation unit to provide an electrical pulse train to a target portion of a vagus nerve, said electrical pulse train comprising a first pulse having a first characteristic during a first time period, said first characteristic comprising at least one of a first amplitude, a first polarity, a first pulse width, and a first pulse shape; and
a controller operatively coupled to said stimulation unit, said controller being adapted to direct said stimulation unit to apply said pulse train having said first pulse to said target portion of the vagus nerve, the controller also being adapted to perform a controlled modification of said first characteristic to generate a second pulse with a second characteristic during a second time period, said second characteristic comprising at least one of a second amplitude different from said first amplitude, a second polarity different from said first polarity, a second pulse width different from said first pulse width and a second pulse shape different from said first pulse shape.

31. The implantable medical device of claim 30, said first characteristic comprising at least two of said first amplitude, said first polarity, said first pulse width, said first pulse shape, and said second characteristic comprising at least two of a second amplitude different from said first amplitude, a second polarity different from said first polarity, a second pulse width different from said first pulse width and a second pulse shape different from said first pulse shape.

32. The implantable medical device of claim 30, said controller being further adapted to perform at least one of a controlled frequency sweep and a random frequency sweep.

33. The implantable medical device of claim 30, said controller being further adapted to perform a controlled modification of said second characteristic to generate a third pulse with a third characteristic during a third time period, said third characteristic comprising at least one of a third amplitude different from said second amplitude, a third polarity different from said second polarity, a third pulse width different from said second pulse width and a third pulse shape different from said second pulse shape.

34. An implantable medical device (IMD) for delivering a tri-phasic stimulation signal to a cranial nerve of a patient, comprising:
a stimulation unit to provide to a target portion of a cranial nerve an electrical pulse comprising a first phase corresponding to a first time period and having a first characteristic, a second phase corresponding to a second time period and having a second characteristic, and a third phase corresponding to a third time period and having a third characteristic; and
a controller operatively coupled to said stimulation unit, said controller being adapted to direct said stimulation unit to apply said electrical pulse to said target portion of said cranial nerve.

35. The implantable medical device of claim 34, said stimulation unit further comprising a phase controller for providing a timing reference for the duration of at least one of said first phase, said second phase and said third phase.

36. The implantable medical device of claim 34, said phase controller further comprising a phase timer for providing said timing reference.

37. The implantable medical device of claim 34, wherein said first characteristic comprises a first polarity, said second characteristic comprises a second polarity opposite said first polarity and said third characteristic comprises a third polarity opposite said second polarity.

38. The implantable medical device of claim 34, wherein said third phase comprises a charge-balance signal for balancing an electrical charge resulting from the electrical pulse from at least one of said first phase and said second phase.

39. The implantable medical device of claim 34, wherein said cranial nerve comprises a vagus nerve.

40. A computer readable program storage device encoded with instructions that, when executed by a computer, performs a method for treating a patient with a tri-phasic stimulation signal from an implantable medical device (IMD), comprising:
applying an electrical pulse to a target portion of a cranial nerve using a pulse generator, said electrical pulse comprising a first phase corresponding to a first time period comprising a first characteristic, a second phase corresponding to a second time period comprising a second characteristic, and a third phase corresponding to a third time period comprising a third characteristic, wherein said first characteristic comprises a first polarity and at least one of a first amplitude, a first pulse width, and a first pulse shape, wherein said second characteristic comprises a second polarity opposite said first polarity and at least one of a second amplitude, a second pulse width, and a second pulse shape, and wherein said third characteristic comprises a third polarity opposite said second polarity and at least one of a third amplitude, a third pulse width and a third pulse shape.
